(19) United States
(12) Patent Application Publication
(10) Pub. No.: US 2025/0288355 A1
(43) Pub. Date: Sep. 18, 2025

(54) SUBJECT-SPECIFIC IMAGE-BASED MULTIMODAL AUTOMATIC 3D PRE-SURGICAL AND REAL-TIME GUIDANCE SYSTEM FOR NEURAL INTERVENTION

(71) Applicant: Sheba Impact Ltd., Ramat-Gan (IL)

(72) Inventors: Abigail LIVNY-EZER, Tel-Aviv (IL); Zion ZIBLY, Herzeliya (IL); David MESIKA, Tal Shachar (IL); Reut Moran RAIZMAN, Rosh HaAyin (IL)

(73) Assignee: Sheba Impact Ltd., Ramat-Gan (IL)

(21) Appl. No.: 18/861,923

(22) PCT Filed: May 1, 2023
(86) PCT No.: PCT/IL2023/050440

§ 371 Date: Oct. 31, 2024

(30) Foreign Application Priority Data
May 1, 2022 (IL) 292663

(51) Int. Cl. A61B 34/10; A61B 5/00; A61B 5/055; A61B 34/20; A61B 90/00; G06T 7/11; G06T 7/33; G06T 17/00; G16H 20/40

(52) U.S. Cl. CPC A61B 34/10; A61B 5/0042; A61B 5/055; G06T 7/11; G06T 7/337; G06T 17/00; G16H 20/40; A61B 2034/105; A61B 2034/107; A61B 2034/2065; A61B 2090/367; A61B 2576/026; G06T 2207/20128; G06T 2207/30016; G06T 2210/22

(57) ABSTRACT

There is provided a method of reconstruction of a target brain structure(s), comprising: reconstructing at least a part of a brain comprising boundaries of brain structures that include the target brain structure(s), wherein the reconstruction is insufficient for parceling of the target brain structure(s) into sub-structures of a same type of gray or white matter, reconstructing and parceling the target brain structure(s) using a reference atlas, segmenting and parceling at least one originating brain structure using the reference atlas, filtering white matter fibers to isolate at least one target white matter tract connecting the originating brain structure(s) and the target brain structure(s), and creating a 3D reconstruction of the target brain structure(s) and the target white matter tract(s), wherein the target white matter tract(s) and the target brain structure(s) are transformed and/or mapped to an anatomical native space.

### RELATED APPLICATION

This application claims the benefit of priority of Israel Patent Application No. 292663 filed on May 1, 2022, the contents of which are incorporated herein by reference in their entirety.

### BACKGROUND

The present invention, in some embodiments thereof, relates to image processing of medical images and, more specifically, but not exclusively, to reconstruction of brain structures based on image processing of medical images.

Stereotactic brain surgery modalities rely on images to guide the surgeon to the exact location in the brain in which to perform a treatment.

### SUMMARY

According to a first aspect, a computer implemented method of reconstruction of at least one target brain structure, comprises, using at least one processor for processing a plurality of images depicting a brain of a subject by: reconstructing at least a part of the brain comprising boundaries of a plurality of brain structures that include the at least one target brain structure, wherein the reconstruction is insufficient for parceling of the at least one target brain structure into sub-structures of a same type of gray or white matter, reconstructing and parceling the at least one target brain structure using a reference atlas, segmenting and parceling at least one originating brain structure using the reference atlas, filtering white matter fibers to isolate at least one target white matter tract connecting the at least one originating brain structure and the at least one target brain structure, and creating a 3D reconstruction of the at least one target brain structure and the at least one target white matter tract, wherein the at least one target white matter tract and the at least one target brain structure are transformed and/or mapped into an anatomical native space.

According to a second aspect, a system for reconstruction of at least one target brain structure, comprises: at least one processor executing a code for processing a plurality of images depicting a brain of a subject by: reconstructing at least a part of the brain comprising boundaries of a plurality of brain structures that include the at least one target brain structure, wherein the reconstruction is insufficient for parceling of the at least one target brain structure into sub-structures of a same type of gray or white matter, reconstructing and parceling the at least one target brain structure using a reference atlas, segmenting and parceling at least one originating brain structure using the reference atlas, filtering white matter fibers to isolate at least one target white matter tract connecting the at least one originating brain structure and the at least one target brain structure, and creating a 3D reconstruction of the at least one target brain structure and the at least one target white matter tract, wherein the at least one target white matter tract and the at least one target brain structure are mapped to an anatomical native space. According to a third aspect, a non-transitory medium storing program instructions for reconstruction of at least one target brain structure, which, when executed by at least one processor, cause the at least one processor to: process a plurality of images depicting a brain of a subject by: reconstructing at least a part of the brain comprising boundaries of a plurality of brain structures that include the at least one target brain structure, wherein the reconstruction is insufficient for parceling of the at least one target brain structure into sub-structures of a same type of gray or white matter, reconstructing and parceling the at least one target brain structure using a reference atlas, segmenting and parceling at least one originating brain structure using the reference atlas, filtering white matter fibers to isolate at least one target white matter tract connecting the at least one originating brain structure and the at least one target brain structure, and creating a 3D reconstruction of the at least one target brain structure and the at least one target white matter tract, wherein the at least one target white matter tract and the at least one target brain structure are mapped to an anatomical native space.

In a further implementation form of the first, second, and third aspects, further comprising:

creating a single combined 3D image by combining the 3D reconstruction of the at least one target brain structure and the at least one target white matter tract with the at least one originating brain structure transformed and/or mapped into the anatomical native space and with a background depicting a structure of the brain defined within the anatomical native space, wherein the single combined 3D image is compatible with medical archive standards for storing and presenting 3D medical images. In a further implementation form of the first, second, and third aspects, further comprising: automatically detecting at least one stereotactic landmark in proximity to and external to the at least one target brain structure, realigning a first image to (right-anterior-superior) RAS space using linear realignment, wherein the at least one stereotactic landmark is automatically detected in the realigned first image and set as an origin of the first image, and aligning a volume of the brain so that the at least one stereotactic landmarks are set on a same axial plane as the origin.

In a further implementation form of the first, second, and third aspects, the at least one stereotactic landmark is selected from a group comprising: anterior commissure (AC), posterior commissure (PC), and third ventricle, and wherein aligning comprises aligning an Inter-Commissural Line (ICL) as an anterior-posterior axis.

In a further implementation form of the first, second, and third aspects, further comprising: wherein the at least one stereotactic landmark is of a first anatomical image in proximity to and external to the at least one target brain structure, and co-registering at least one second image to an aligned first anatomical image using the at least one stereotactic landmark to obtain same image dimensions and/or orientation.

In a further implementation form of the first, second, and third aspects, further comprising feeding an aligned and centered anatomical image as input into a reconstruction process that reconstructs the part of the brain, wherein the part of the brain comprises a whole brain.

In a further implementation form of the first, second, and third aspects, further comprising marking a location indicating the at least one stereotactic landmark on the 3D reconstruction.

In a further implementation form of the first, second, and third aspects, the at least one target brain structure comprises at least one ventral intermediate nucleus (VIM) of a thalamus, and the at least one originating brain structure comprises at least one Dentate nucleus.

In a further implementation form of the first, second, and third aspects, further comprising segmenting the at least one target brain structure according to a second image registered to the first image, wherein the second image is captured using a second protocol different than a first protocol used to capture the first image.

In a further implementation form of the first, second, and third aspects, segmenting comprises segmenting at least one non-target brain structure located in proximal to and external to the at least one target brain structure.

In a further implementation form of the first, second, and third aspects, the at least one target brain structure and the at least one non-target brain structure are of a same matter type and are sub-structures within a main structure.

In a further implementation form of the first, second, and third aspects, the non-target brain structure comprises a ventral caudal (VC) nucleus or nearby brain tissue that is at risk for unintended damage during treatment of a VIM.

In a further implementation form of the first, second, and third aspects, further comprising cropping the at least one non-target brain structure and the at least one target brain structure to create cropped images, reslicing the cropped images to dimensions of an aligned first image, and overlaying the resliced cropped images upon the first image, wherein boundaries of the at least one non-target brain structure and the at least one target brain structure are depicted in the overlay.

In a further implementation form of the first, second, and third aspects, segmenting at least one originating brain structure comprises isolating and segmenting a main brain structure that includes the originating brain structure therein from the anatomical scan, computing a transformation matrix for registering the isolated main brain structure to a space of an atlas, inversely transforming the atlas using the transformation matrix into a structural reconstruction space comprising the segmented and parcellated at least one originating brain structure.

In a further implementation form of the first, second, and third aspects, further comprising merging the reconstruction of the part of the brain with the segmented at least one target brain structure and the segmented at least one originating brain structure to create a merged image.

In a further implementation form of the first, second, and third aspects, creating the 3D reconstruction comprises overlaying the at least one target brain structure, and overlaying at least one of: the at least one originating brain structure, and the at least one target white matter tract, on a background anatomical scan in the anatomical native space.

In a further implementation form of the first, second, and third aspects, the at least one target brain structure comprises a VIM of a thalamus, and further comprising treating the subject for essential tremor by applying focused ultrasound to the VIM.

In a further implementation form of the first, second, and third aspects, further comprising feeding the 3D reconstruction into a guidance system for neural intervention for image guided treatment of the at least one target brain structure guided by the 3D reconstruction.

In a further implementation form of the first, second, and third aspects, the plurality of images comprise a first image captured using a first protocol, a second image captured using a second protocol different than the first protocol, and a third image captured using a third protocol different than the first protocol and the second protocol.

In a further implementation form of the first, second, and third aspects, the first protocol is designed for contrast between gray matter and white matter of the brain.

In a further implementation form of the first, second, and third aspects, the first image a comprises a T1 weighted MRI image, the second image comprises a T2 weighted MRI image, and the third image comprises a diffusion-weighted image.

In a further implementation form of the first, second, and third aspects, the second protocol is designed for segmentation of the at least one brain structure.

In a further implementation form of the first, second, and third aspects, the at least one target brain structure comprises at least one VIM of a thalamus, the at least one originating brain structure comprises at least one Dentate nucleus, and the at least one white matter tract comprises a Dento-Rubro Thalamic Tract (DRTT) extending from the at least one Dentate nucleus to the VIM.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system for annotation and/or reconstruction of one or more target brain structures, in accordance with some embodiments of the present invention;

FIG. 2 is a flowchart of a method of annotation and/or reconstruction of one or more target brain structures, in accordance with some embodiments of the present invention;

FIG. 3 is a schematic depicting a slice of a 3D T1 weighted MRI scan with automatically identified landmarks, in accordance with some embodiments of the present invention;

FIG. 4 is a schematic depicting a reorientation of the slice of the 3D T1 weighted MRI scan, in accordance with some embodiments of the present invention;

FIG. 5 is a schematic of a whole brain reconstruction, that includes segmentation and parcellation of brain structures, in accordance with some embodiments of the present invention;

FIG. 6 is a schematic of a thalamic parcellation, in accordance with some embodiments of the present invention;

FIG. 7 is a schematic of an image depicting both a cerebellum with dentate nuclei and thalamus with nuclei, in accordance with some embodiments of the present invention;

FIG. 8 is a schematic depicting isolated white matter fibers of the DRTT connecting between a dentate nucleus of the cerebellum to a VIM nucleus of the thalamus, in accordance with some embodiments of the present invention;

FIG. 9 is a schematic of an exemplary 3D reconstruction, in accordance with some embodiments of the present invention;

FIG. 10 is a schematic of subject specific masks of the VIM and VC, and of the DRTT registered and merged together with a T1 weighted scan, in accordance with some embodiments of the present invention;

FIG. 11 includes graphs presenting results of surgical parameters in an experiment comparing classic stereotactic targeting and personalized integrative targeting, in accordance with some embodiments of the present invention;

FIG. 12 includes graphs presenting results of adverse effects in an experiment comparing classic stereotactic targeting and personalized integrative targeting, in accordance with some embodiments of the present invention;

FIG. 13 is a schematic depicting preliminary data of pre-and post-surgical scans which undergone the subject-specific 3D reconstruction approaches described herein, in accordance with some embodiments of the present invention.

### DETAILED DESCRIPTION

The present invention, in some embodiments thereof, relates to image processing of medical images and, more specifically, but not exclusively, to reconstruction of brain structures based on image processing of medical images.

As used herein, the term segmentation (or segmenting or other variations thereof) refers to an image processing stage that discriminates and/or separates objects in an image based on pixel characteristics, for example, a segmentation processes that marks boundaries that separate between brain tissues such as white and gray matter. Segmentation may be implemented, for example, by a segmentation neural network that may be trained on images of the brain marked with ground truth segmentation, and/or approaches such as intensity values and/or based on identified features. As used herein, the term parcellation (or parceling or other variations thereof) relates to an image processing stage that divides the selected objects (e.g., segmented) into sub-regions within a specific matter, for example, nuclei within the thalamus. Since the sub-regions are the same matter type as the selected (e.g., segmented) region, standard segmentation methods usually cannot be applied, since the sub-regions cannot be accurately visually differentiated, even manually such as by a trained human expert, and/or automatically such as by a neural network or other segmentation process. Parcellation may be done, for example, by an atlas containing labels of regions. Parcellation may be done by anatomical and/or functional division. It is noted that parcellation using an atlas is therefore non-specific to the individual subject, for example, when a general atlas of general anatomical structure is used, anatomical variations (e.g., size, location) are not accurately captured.

The terms fiber(s) and tract(s) are sometimes used interchangeably herein.

An aspect of the present invention relates to systems, methods, computing devices, and code (i.e., stored on a data storage device and executable by at least one processor) for reconstruction of one or more target brain structures that are of a certain type and in proximity to other brain structures of a same type, for example, the target and other brain structures are both gray matter, or both white matter, for example, nuclei of the thalamus such as a ventral intermediate (VIM) nucleus of the thalamus, such as for targeted treatment of essential tremor. Such brain structures which are of a same type and located in proximity to each other cannot be automatically differentiated using existing approaches, since they are not well defined visually and cannot be visually delineated well enough. One or more images of at least a part of the brain are processed to generate the reconstruction. A part of the brain, optionally the whole brain, that includes boundaries of multiple brain structures is reconstructed. The reconstructed portion includes the target brain structure(s), but is insufficient for parceling of the target brain structure(s) into sub-structures of a same type of gray or white matter. The target brain structure(s) is reconstructed and/or parcellated, optionally using a reference atlas. One or more originating brain structures are segmented and/or parcellated from the anatomical image and/or from the reconstruction of the part of the brain using a reference atlas (which may be the same reference at last used to reconstruct and/or parecellate the target brain structure and/or a different reference atlas). A tractogram of at least the part of the brain, optionally the whole brain, may be created.

The white matter fibers of the tractogram may be filtered to isolate one or more target white matter tracts that connect the originating brain structure and the target brain structure. The intersection between the filtered white matter fibers and the target brain structure provides an accurate indication for the target location within the target brain structure. The specific set of white matter fibers that connect to the target brain structure is identified based on the fact that those white matter fibers also connect to the originating brain structure. Identifying the white matter fibers may improve the location of the target brain structure(s) from the location defined by the reference atlas to the personalized location in the anatomy of the subject, based on the fact that the white matter fibers physically connect to the target brain structure(s). A 3D reconstruction that includes at least the target brain structure and the target white matter tract may be computed. The target white matter fiber(s) and the target brain structure(s) may be mapped to an anatomical native space. The 3D reconstruction, optionally within the anatomical native space, provides an accurate location of the target brain structure of the subject, which may be used, for example, for accurate targeted treatment of the target brain structure and/or reducing risk of inadvertently targeting other brain structures in proximity to the target brain structure. Use of the 3D reconstruction may shorten duration of the treatment of the target brain structure (e.g., surgery). The anatomical native space of the 3D reconstruction may enable providing simultaneously accurate locations of the white matter fiber(s) and the target brain structure(s), optionally be enabling determining the relative location of the white matter fiber(s) and the target brain structure(s) depicted within the anatomical native space.

In at least some embodiments described herein, the white matter fiber(s) and/or the target brain structure(s) and/or the originating brain structure(s) and/or other structures described herein, may be transformed and/or mapped into the native anatomical space, for example, the T1 space of the MRI machine that captured the images used for the 3D reconstruction, and/or any other space that may be defined and/or selected, such as other native anatomical spaces of other imaging devices capturing images used for the 3D reconstruction and/or other spaces. The transformation and/or mapping into the native anatomical space may be in contrast, for example, to obtaining two different images into two different spaces, such as one image of the target brain structure(s), and another image of the originating brain structure(s), which are in different spaces, and therefore cannot be used to accurately determine the relative locations of the target brain structure(s), and the originating brain structure(s).

In an exemplary use case described herein, the target brain structure is the VIM of the thalamus, the originating brain structure is the Dentate nucleus of the cerebellum, and the target white matter fibers are the Dento-Rubro Thalamic Tract (DRTT) extending from the Dentate nucleus through the red nucleus of the midbrain to the VIM of the thalamus. The VIM may be targeted, for example, by image-guided focused ultrasound for treatment of essential tremor.

It is noted that the exemplary case of the target brain structure being the VIM of the thalamus for targeted treatment for essential tremor is provided as a not necessarily limiting example. Embodiments described herein may be used for other gray and/or white matter brain structures which are difficult to accurately demarcate. Such brain structures may be in proximity to other brain structures of a same type, which may make them particularly difficult to accurately demarcate. The other brain structures may be, for example, other nuclei, specific white matter tracts, and pathological structures such as tumors. The other brain structures may be targeted for treatment of other medical conditions. For example, treatment of brain tumors, epilepsy, Alzheimer's, other movement disorders (e.g., deep brain stimulation) and the like.

At least some embodiments described herein provide a solution to the technical problem(s) described herein, and/or improve the technology described herein, by reconstructing at least a part of the brain, optionally the whole brain. The reconstruction is insufficient for parceling of the target brain structure into sub-structures of a same type of gray or white matter, for example, the reconstruction is insufficient for differentiating between the VIM of the thalamus and other nearby nuclei that may suffer collateral damage during treatment of the VIM. The reconstruction is used for segmenting and/or parceling one or more originating brain structure(s), that are connected to the target brain structures, for example, the originating brain structure(s) are gray matter connected to the target brain structure(s) via white matter tracts. In the exemplary case, the originating brain structure(s) is the Dentate nucleus of the cerebellum, which is connected to the VIM via the DRTT. The target brain structure is reconstructed and/or parcellated, optionally using a reference atlas, which provides an initial first approximate location, since the target brain structure cannot be accurately visually delineated on its own, as described herein. The target white matter fibers connecting the target and originating brain structures are filtered and isolated. A 3D reconstruction that includes at least the target brain structure, the originating brain structure, and the target white matter tract, is generated. The target brain structure and/or the originating brain structure, and/or the target white matter tract may be transformed and/or mapped to the anatomical native space.

The 3D reconstruction, optionally within the anatomical native space, provides an accurate location of the target brain structure(s) based on the identified white matter fibers, which may correct the location of the target brain structure(s) from the location defined by the reference atlas to the personalized location in the anatomy of the subject. The higher accuracy in the location of the target brain structure may be based on the fact that the white matter fibers physically connect to the target brain structure(s), where the white matter fibers and the target brain structure(s) may be represented within the anatomical native space.

At least some embodiments described herein address the technical problem of improving accuracy of image-guided treatments of the brain, such a focused ultrasound and/or stereotactic radiosurgery. At least some embodiments described herein improve the technical field of image-guided treatments of the brain. At least some embodiments described herein improve upon existing approaches for image-guided treatments of the brain. At least some embodiments described herein provide a solution to the technical problem, and/or improve the technical field, and/or improve over prior approaches, by providing a fully automatic approach for personalized reconstruction of a target brain region, that is used to accurately guide the treatment by an image guided modality.

Examples of image-guided treatments include focused ultrasound, stereotactic radiosurgery, and implantation of electrodes (e.g., deep brain stimulation (DBS)). Examples of treatments using the image-guided modalities include: brain tumor removal, DBS, electrode, magnetic resonance imaging-guided focused ultrasound (MRgHIFU) thalamotomy, epilepsy treatment, Alzheimer's treatment, and the like. One example of image-guided treatment of the brain which is now described in additional detail is MRgHIFU thalamotomy, which is a non-invasive, image-guided procedure in which thalamic tissue is ablated with submillimeter precision. It is currently FDA-approved for essential tremor (ET), in particular drug resistant ET, and tremor dominant Parkinson's disease and is performed unilaterally. ET is the most common cause of action tremor, with an estimated prevalence worldwide of 1 percent overall and approximately 5 percent in adults over the age of 60 years. The tremor rate is evaluated by a neurologist using a clinical rating scale for tremor (CRST), and an evaluation of simple motor functionalities as drawing tasks and drinking from a cup. During MRgHIFU, concentrated ultrasonic waves cause local thermal heating to intracranial tissue with submillimeter precision. Temperature level and duration of exposure determine the resulting lesion size, for example, as described with reference to Fiani B, Lissak I A, Soula M, et al. The Emerging Role of Magnetic Resonance Imaging-Guided Focused Ultrasound in Functional Neurosurgery. Cureus 2020; 12. doi: 10.7759/cureus.9820, incorporated herein by reference in its entirety. When successful, the procedure significantly reduces or eliminates the tremor (e.g., achieves contralateral tremor suppression) with improvement of patients' manual function and quality of life. In the case of essential tremor, the target of the ablation is defined as the entry-zone of the Dento-Rubro Thalamic Tract (DRTT) into the VIM nucleus, for example, as described with reference to Gallay M N, Jeanmonod D, Liu J, et al. Human pallidothalamic and cerebellothalamic tracts: Anatomical basis for functional stereotactic neurosurgery. Brain Struct Funct 2008; 212: 443-63. doi: 10.1007/s00429-007-0170-0 incorporated herein by reference in its entirety. Histologically, the VIM size is approximately 4 mm in the anterior-posterior dimension, 4 mm medial-laterally, and 6 mm dorsal-ventrally, representing 0.5-2.0% of the total thalamic volume, for example, as described with reference to Hirai T, Ohye C, Nagaseki Y, et al. Cytometric analysis of the thalamic ventralis intermedius nucleus in humans. J Neurophysiol Published Online First: 1989. doi: 10.1152/jn.1989.61.3.478 incorporated herein by reference in its entirety. Advanced MR sequences, such as quantitative susceptibility mapping (QSM), fast gray matter acquisition T1 inversion recovery (FGATIR) and white matter attenuated inversion recovery (WAIR), that improve the ability to image the VIM region, have become popular. However, detecting the exact coordinates for the ablation targeting using these sequences is not yet feasible, and these methods have not shown significant reliability and accuracy to serve as the primary method for VIM targeting, for example, as described with reference to Najdenovska E, Alemán-Gómez Y, Battistella G, et al. In-vivo probabilistic atlas of human thalamic nuclei based on diffusion-weighted magnetic resonance imaging. Sci Data 2018;5:1-11. doi: 10.1038/sdata.2018.270 incorporated herein by reference in its entirety.

Existing approaches of targeting can be divided into two distinct categories: direct and indirect targeting, where indirect targeting is based on stereotactic atlas or driven landmarks, and direct targeting involves white matter reconstruction of the DRTT in diffusion native space. The indirect targeting approaches (e.g., using landmarks, atlas based parcellation) used to find the target brain structure(s) and the direct target approaches (e.g., reconstruction of the DRTT) used to find the target white matter fibers are considered as alternatives, and represent two mutually exclusive approaches, where the physician performing the operation selects one of them in order to guide to the target brain region. Traditionally, only one of the indirect or direct targeting approaches is used. Standard approaches do not combine the indirect and direct target approaches.

In contrast, at least some embodiments described herein combine the indirect target approach and the direct target approach into a combined method and/or system and/or device and/or code stored on a data storage device for executing by one or more processors, by generating a 3D reconstruction that includes the target white matter fibers (e.g., DRTT) found using the direct targeting approach and the target brain structure found using the indirect targeting, transformed and/or mapped into an anatomical native space. Including the target white matter fibers and the target brain structure (and/or other structures described herein) simultaneously within the anatomical native space may enable accurately determining the relative locations of the structures, and/or accurate localization of the individual structures, for example, for treatment as described herein. A recent comparative review compared predicted direct and indirect based coordinates to the actual lesion coordinates that showed relief in symptoms. Direct targeting showed inferior error values on the Right-Left (RL) and Anterior-Posterior (AP) coordinates and higher error values on the Superior-Inferior (SI) coordinates in relation to lesion final location compared to indirect targeting, for example, as described with reference to Bruno F, Catalucci A, Varrassi M, et al. Comparative evaluation of tractography-based direct targeting and atlas-based indirect targeting of the ventral intermediate (Vim) nucleus in MRgFUS thalamotomy. Sci Rep 2021; 11: 1-9. doi: 10.1038/s41598-021-93058-2 incorporated herein by reference in its entirety.

To date, most neurosurgeons conducting MRgHIFU procedures determine the surgical target based on stereotactic, atlas driven, indirect targeting commonly using T1-weighed and proton density (PD) scans. These scans have a very low intrinsic contrast between the target (VIM), adjacent tissue of the thalamus and its surroundings (mainly, sensory ventral-caudal (VC), ventralis-oralis posterior (Vop) and internal capsule), for example, as described with reference to Abosch A, Yacoub E, Ugurbil K, et al. An assessment of current brain targets for deep brain stimulation surgery with susceptibility-weighted imaging at 7 tesla. Neurosurgery 2010; 67: 1745-56. doi: 10.1227/NEU.0b013e3181f74105 incorporated herein by reference in its entirety. Thus, MRgHIFU targeting is currently planned using stereotactic coordinates based on manual pinpointing of the following brain landmarks: Anterior Commissure (AC), Posterior Commissure (PC), and the lateral wall of the third ventricle contralateral to the treated limb. The direct stereotactic targeting method described in the literature and mostly used in practice suggests setting the PC as the image origin, moving 25% of the length of the Inter-Commissural Line (ICL) forward along the AP axis anterior to the PC, 10-11 mm from the lateral wall of the third ventricle contra-lateral to the tremorous limb, and 0-1 mm superior or inferior to the PC, for example, as described with reference to Spiegelmann R, Nissim O, Daniels D, et al. Stereotactic targeting of the ventrointermediate nucleus of the thalamus by direct visualization with high-field MRI. Stereotact Funct Neurosurg Published Online First: 2006. doi: 10.1159/000092683 incorporated herein by reference in its entirety.

The scans described above have a very low intrinsic contrast between the target (VIM), adjacent nuclei and adjacent white-matter tracts. Thus, targeting is planned using manual pinpointing of the specific brain landmarks. Since the size and parcellation in individual brains can differ from patient to patient, this method of targeting leads to a procedure of trial and error during surgery. Finally, the variance of target ablations can cause damage to adjacent nuclei, leading to adverse side effects such as ataxia, gait disturbance, damage to senses of smell and taste and other adverse side effects.

At least some embodiments described herein accurately reconstruct the target brain structure, for example, the VIM, which may be used for a personalized MR-based multimodal automatic surgical guidance system, which determines landmark locations as well as provides detailed visual description of the surgical target, including 3D models of segmented tissues and adjacent fibers. Embodiments described herein use to reconstruct target brain structure for pre-surgical as well as real-time planning. Embodiments described herein using the reconstructed target brain structure provide one or more of the following potential technical improvements: lesion location variance is reduced, and/or surrounding brain (e.g., thalamic) tissue is less likely to be damaged. In turn this may lead to higher procedure success rates and/or long-term efficacy, reduce time in surgery and reduce unwanted adverse effects.

At least some embodiments described herein improve accuracy of image-guided treatments of the brain, such a focused ultrasound and/or stereotactic radiosurgery. At least some embodiments described herein shorten duration of treatment of the target region of the brain, such as surgery, optionally using focused ultrasound and/or stereotactic radiosurgery, for example, as described in the Examples section below. At least some embodiments described herein improve the technical field of image-guided treatments of the brain. At least some embodiments described herein improve upon existing approaches for image-guided treatments of the brain. At least some embodiments described herein provide a solution to the technical problem, and/or improve the technical field, and/or improve over prior approaches, by providing a fully automatic approach for accurate reconstruction of a target brain region, that is used to accurately guide the image-guided treatment.

At least some embodiments described herein address the technical problem of delineating brain structures that are difficult or impossible to detect on medical images using standard approaches, for example, nuclei within gray matter, such as nuclei within the thalamus, and/or specific white matter fibers within a larger set of white matter fibers. At least some embodiments described herein improve the technical field of image processing of medical images, by delineating brain structures that are difficult or impossible to detect on medical images using standard approaches, for example, nuclei within gray matter, such as nuclei within the thalamus, and/or specific white matter fibers within a larger set of white matter fibers. At least some embodiments described herein improve upon existing approaches for delineating brain structures that are difficult or impossible to detect on medical images, such as standard indirect and/or direct methods described above, which are manual requiring demarcation by an expert user (e.g., neurosurgeon) and/or not accurate.

At least some embodiments described herein address the technical problem and/or medical problem of reducing number of sonications needed to examine the location of optimal target coordinates, which in turn will lead to reduced length of surgery and thus reduce patient's inconvenience and/or reducing unwanted side effects due to inaccurate ablation location and/or improving the course of the brain surgery by minimizing changes in tissue molecular structure along the ultrasonic ray routes which in turn, prevents reaching ideal ablation temperatures. At least some embodiments described herein improve brain surgery by reducing number of sonications needed to examine the location of optimal target coordinates, which in turn will lead to reduced length of surgery and thus reduce patient's inconvenience and/or reducing unwanted side effects due to inaccurate ablation location and/or improving the course of the brain surgery by minimizing changes in tissue molecular structure along the ultrasonic ray routes which in turn, prevents reaching ideal ablation temperatures. At least some embodiments described herein improve upon prior approaches, by reducing number of sonications needed to examine the location of optimal target coordinates, which in turn will lead to reduced length of surgery and thus reduce patient's inconvenience and/or reducing unwanted side effects due to inaccurate ablation location and/or improving the course of the brain surgery by minimizing changes in tissue molecular structure along the ultrasonic ray routes which in turn, prevents reaching ideal ablation temperatures.

Since significant variability exists in the location of thalamic nuclei and the size and parcellation in individual brains can differ from patient to patient, for example, as described with reference to Ashkan K, Blomstedt P, Zrinzo L, et al. Variability of the subthalamic nucleus: The case for direct MRI guided targeting. In: British Journal of Neurosurgery. 2007. doi: 10.1080/02688690701272240 incorporated herein by reference in its entirety. As such, such standard approaches of targeting may lead to a procedure of trial and error during surgery. Currently, MRgHIFU surgery is conducted by first using low energy sonications on the coordinates initially planned as the target. If a relief in tremor is established and produces transient clinical effects the surgeon will apply more sonications with higher energy until eventually the temperature rises above 55 Celsius where an ablation of the tissue occurs, leaving permanent damage to the brain tissue. If the coordinates planned as the target do not yield a significant relief in tremor the surgeon will try to optimize the target by moving on one or more of the three axes in search for the desired effect. Finally, additional testing and target optimization may increase operative times and potentially affect the tolerability of the procedure. Prolonged sonication phase with multiple target movements, results in longer procedural durations with elevated risk of thrombolytic effects, for example, as described with reference to Lipsman N, Schwartz M L, Huang Y, et al. MR-guided focused ultrasound thalamotomy for essential tremor: A proof-of-concept study. Lancet Neurol Published Online First: 2013. doi: 10.1016/S1474-4422 (13) 70048-6 incorporated herein by reference in its entirety. This standard process of optimizing the target may lead to excessive surgery time, enlarging the patient's discomfort. Finally, the variance of target ablations due to inter-subject variability can also cause damage to adjacent nuclei, leading to adverse side effects such as ataxia, gait disturbance, sensory deficits or motor weakness and other adverse effects, for example, as described with reference to Sinai A, Nassar M, Eran A, et al. Magnetic resonance-guided focused ultrasound thalamotomy for essential tremor: a 5-year single-center experience. J Neurosurg 2020;133:417-24. doi: 10.3171/2019.3.JNS19466, and/or Boutet A, Ranjan M, Zhong J, et al. Focused ultrasound thalamotomy location determines clinical benefits in patients essential tremor. Brain Published Online First: 2018. doi: 10.1093/brain/awy278, incorporated herein by reference in its entirety.

At least some embodiments described herein address the technical problem and/or medical problem of manual annotation of medical image for image guided treatment of the brain, by providing a fully automated approach for accurate detection of target brain structures, even when such brain structures are in proximity to similar looking brain structures, for example, one type of nucleus next to other nuclei, for example, VIM in the thalamus next to other nuclei of the thalamus. At least some embodiments described herein improve the technical field of medical image processing, by providing a fully automated approach for accurate detection of target brain structures. At least some embodiments described herein improve upon existing approaches of manual delineation of target brain structures, by providing a fully automated approach. The fully automated approach provides one or more of the following advantages over manual approaches: faster (manual demarcation may take a long time), many users with varying skill levels may perform it (manual demarcation can only be done by very specialized and trained healthcare personnel such as neuroradiologists and neurosurgeons), and high accuracy and/or objective (manual demarcation is subjective and less accurate). The automated approaches described herein are not simply an automation of a manual process, but involve image processing computations that have no manual counterpart and cannot be performed manually by a human in their head or using pencil and paper.

At least some embodiments described herein provide one or more of the following technical features and/or potential advantages:
Multimodality and Integration—At least some embodiments use 3 common types of MRI scans: 3DT1 weighted, T2-weighted and DTI of 32 directions. All 3 sequences are relatively short, with overall scanning time of about 15 minutes (approximately 5 minutes per protocol). The multimodality integrates information from the direct and indirect methods, from gray-matter structures to white-matter tracts, demonstrated on the subject-space, in order to validate the target coordinates while reducing error factors.
Cross MR platform—At least some embodiments are not segregated to a specific MRI vendor or magnetic field strength (1.5 T and above will suffice for an accurate targeting). As such, data from different centers, different hardware settings and/or different scanning protocols may be used.
Automaticity-At least some embodiments obviate the need of setting the image landmarks, calculating the distances and setting targets by the neurosurgeon. In the case of probabilistic DWI targeting, and DRTT reconstruction, the masking tools may be atlas to subject based and as such, make the manual user (e.g., radiologist) work of marking areas such as VIM and Dentate nucleus on presurgical scans redundant.
Efficacy—At least some embodiments reduce MR/OR time by reducing the sonication phase in which the optimal target is searched for. At least some embodiments prevent changes in SDR that emerge due to multiple sonications, resulting in lower ablation temperature which leads to compromised surgical outcome.
Subject specific, tailor-maid surgical practice—At least some embodiments combine (e.g., DTI based) direct targeting in diffusion native space alongside atlas based parcellation of brain structures (e.g., thalamic nuclei) in other images (e.g., T2 weighted scan). The reconstructed outputs (e.g., brain structures) are not registered to a template space but are transformed and/or mapped in the subject's space (e.g., the anatomical native space is in the subject's space) to avoid deviations arise from individual differences in brain's size, shape, SDR and human judgement bias.
Safety—At least some embodiments act as decision support tool for the neurosurgeon and suggest the location of the surgical target as processed and presented over common structural images or any other registered structural image to the choice of the physician (e.g., T1,T2, PD, and the like). At least some embodiments present brain structures in proximity to the target structure (e.g., nuclei adjacent to the VIM), for example, to reduce or prevent unwanted damage and thus reduce chance for emergence of adverse effects such as ataxia, gait disturbance and so on.

At least some embodiments described herein improve upon prior approaches, by accurately determining the personalized location of the target brain structure for each subject, for example:
Using detailed visual description of the white matter fibers, including 3D models of segmented tissues and/or adjacent fibers, to identify the location of the target brain structure, based on the fact that the white matter fibers physically connect the originating and target brain structures. This is in contrast to other approaches that compute the location of the target brain structure using mathematical equations, which are less accurate and/or not personalized to the subject.
Fully automatic approaches, in contrast to other approaches that require manual input from a user, for example, manual definition of seeds to draw the white matter tracts.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

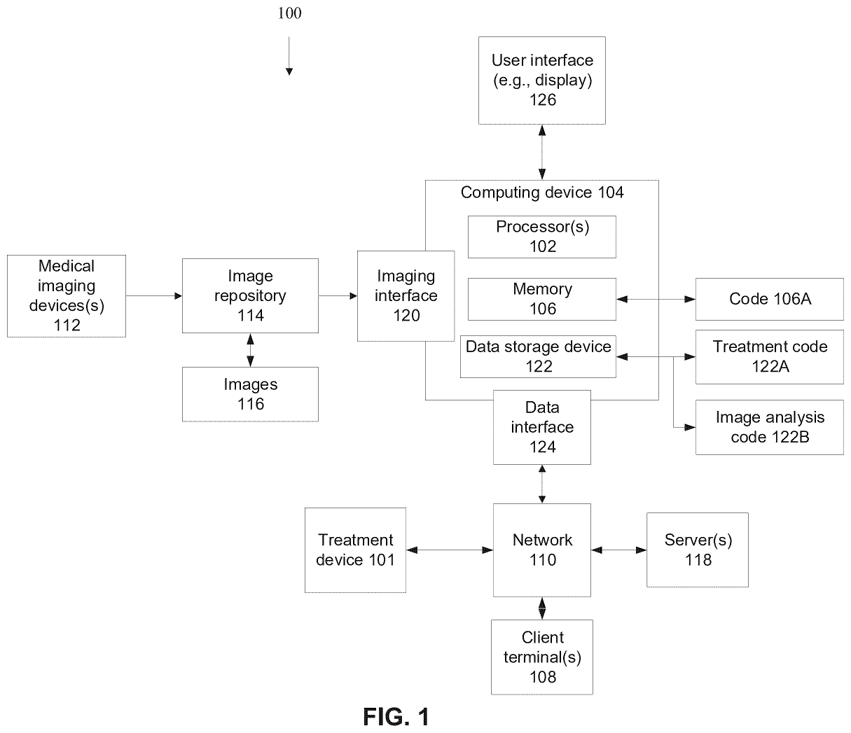

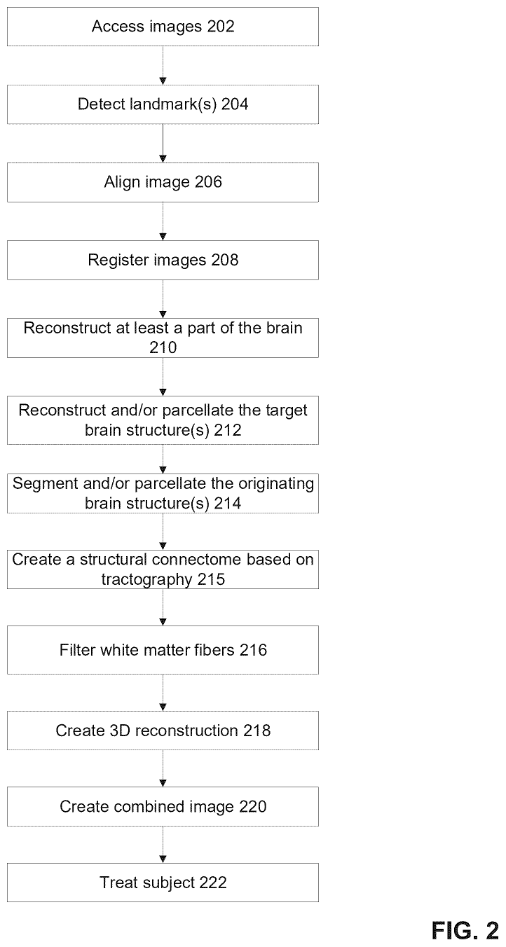

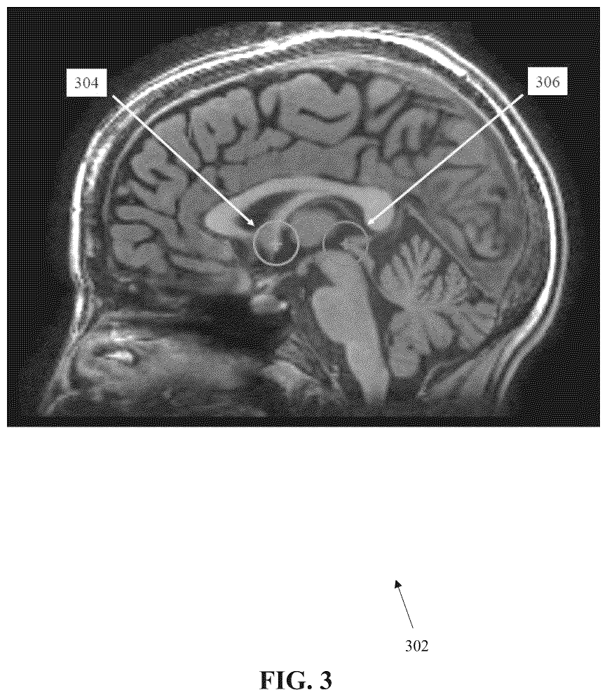

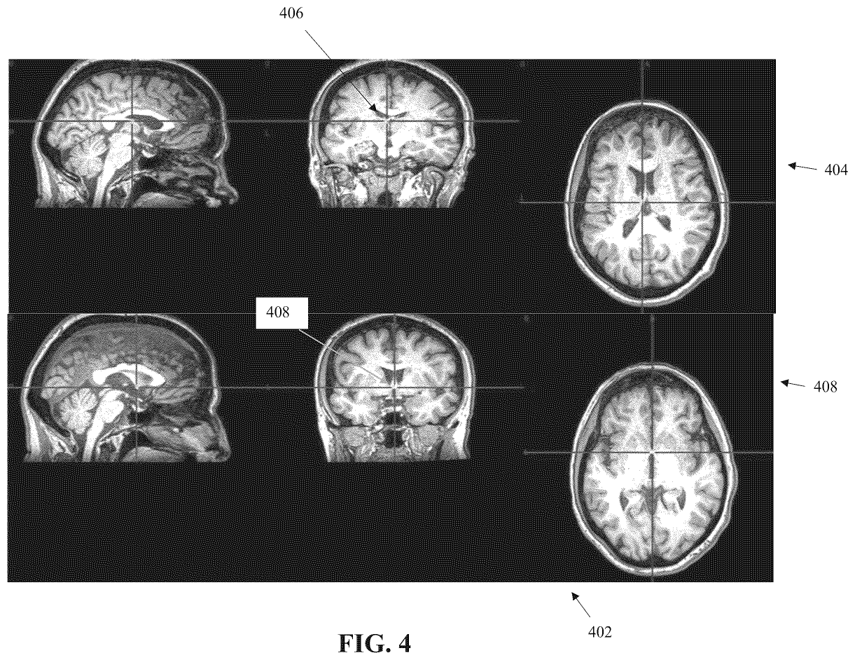

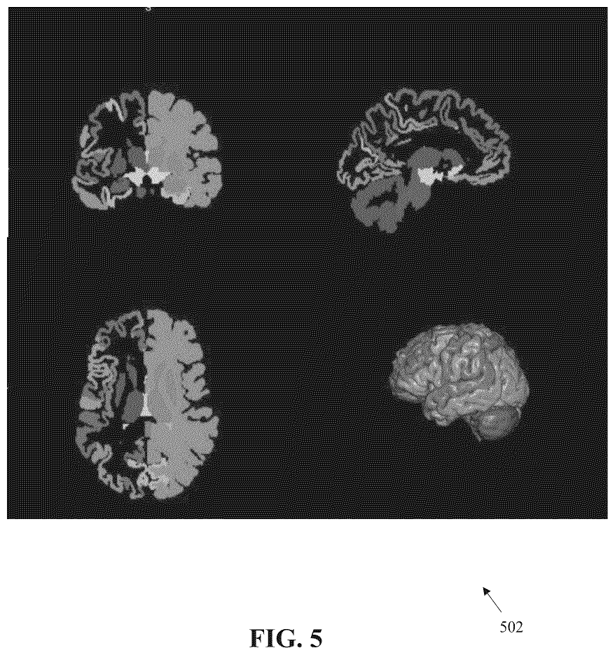

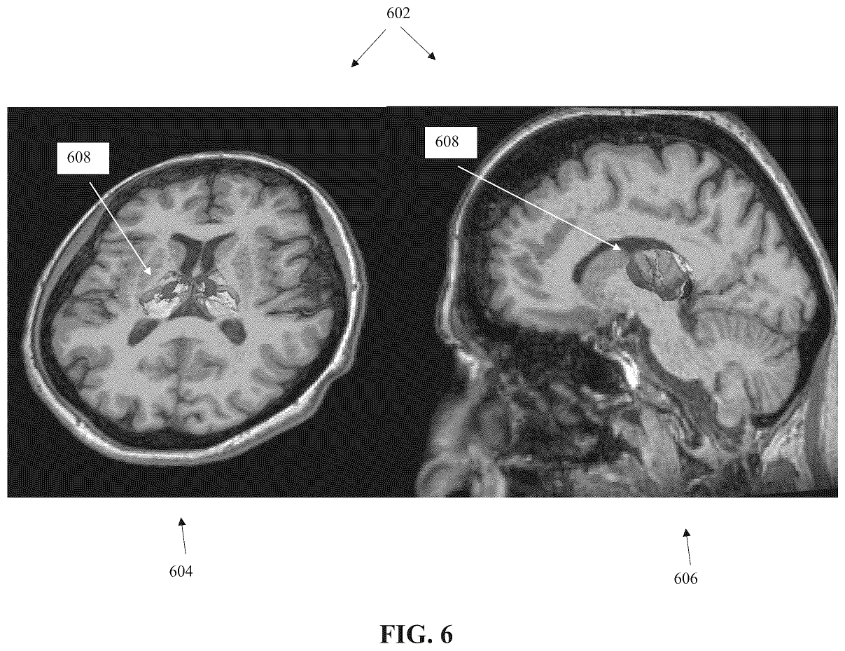

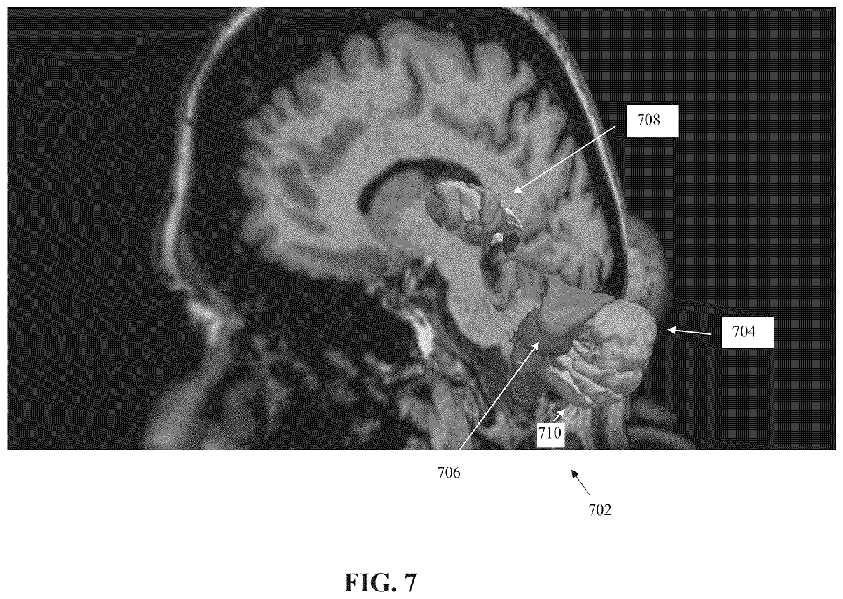

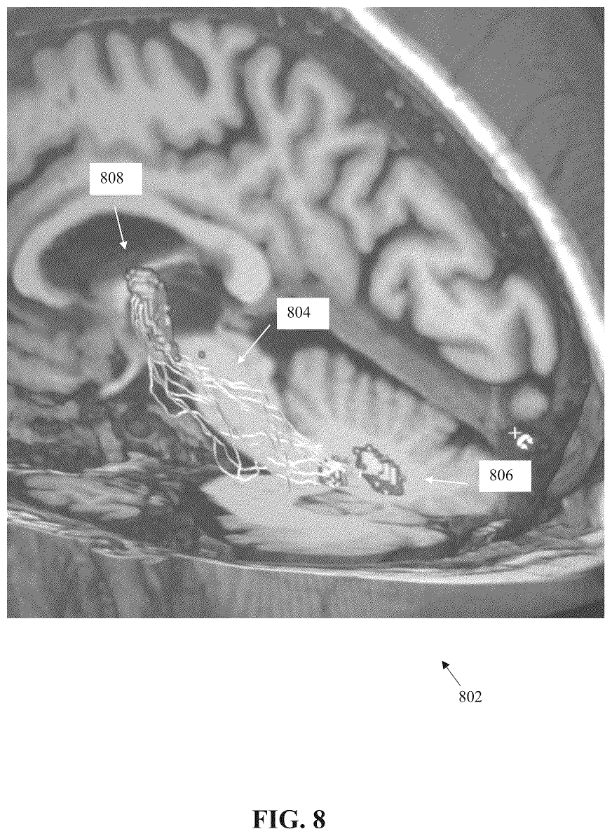

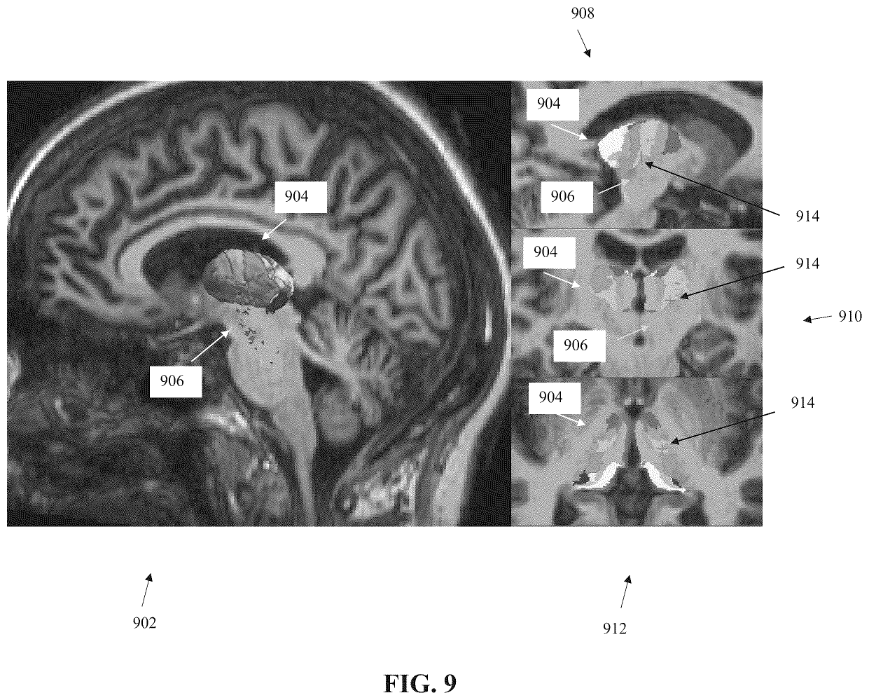

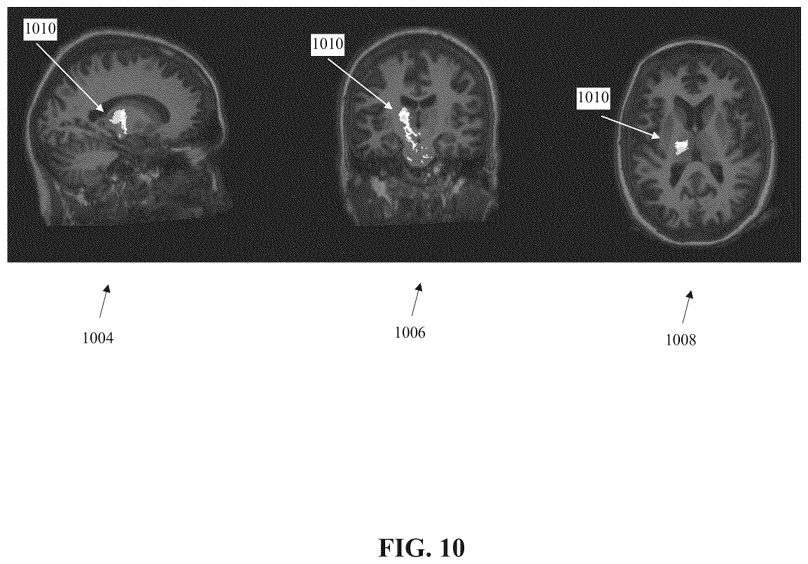

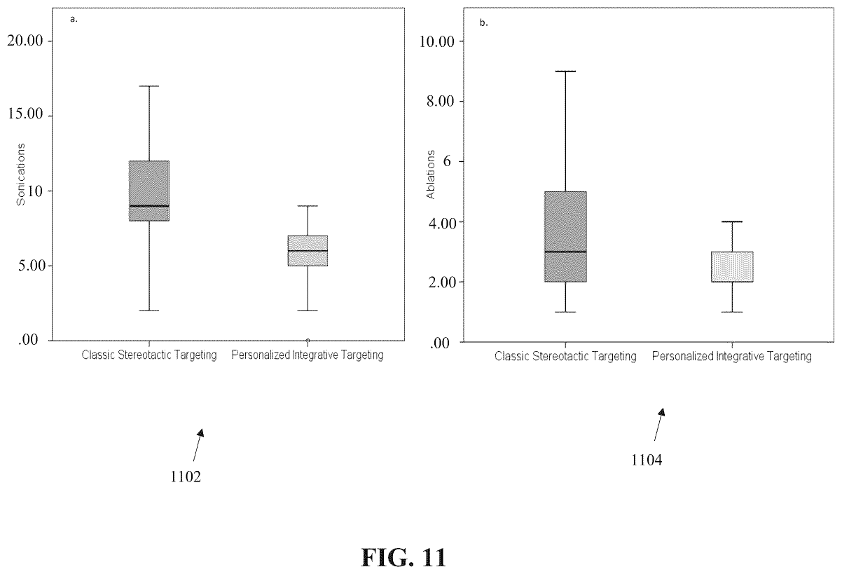

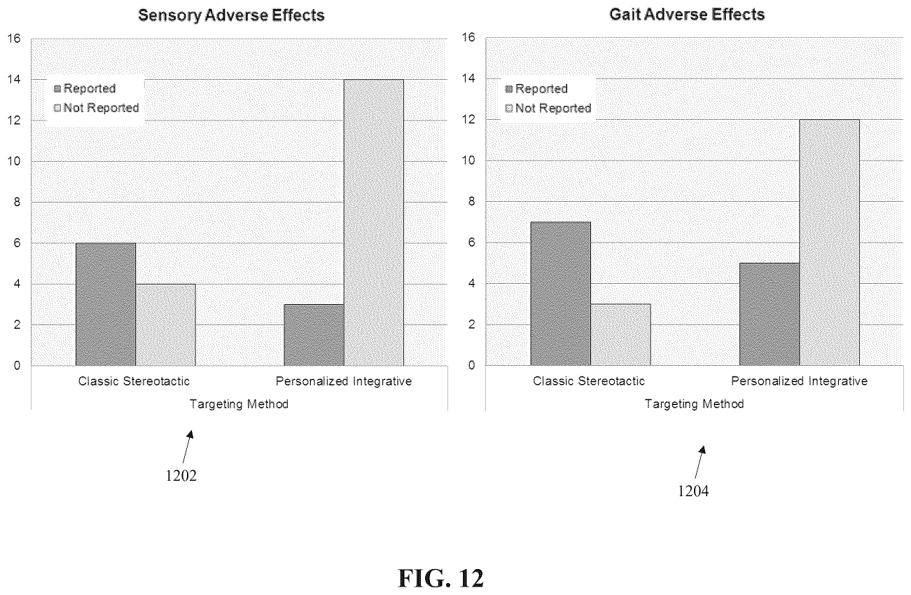

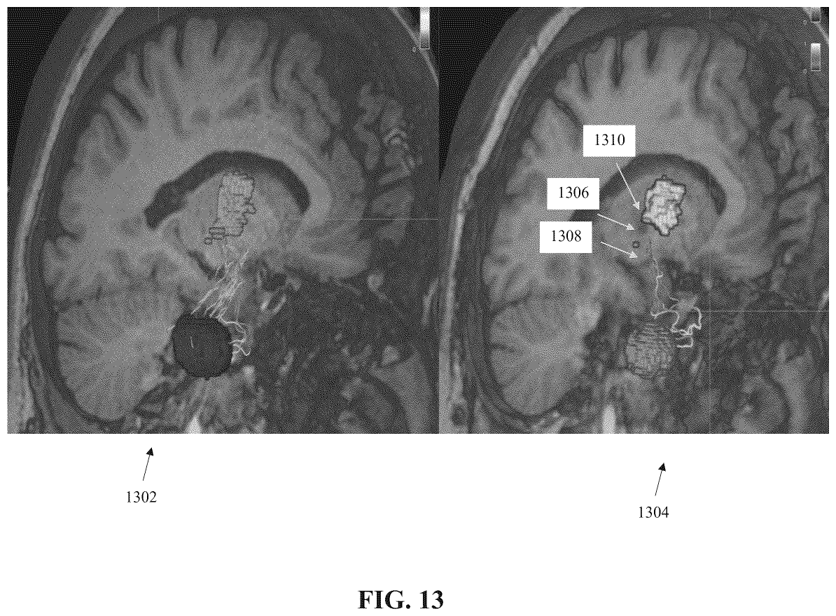

Reference is now made to FIG. 1, which is a block diagram of components of a system for annotation and/or reconstruction of one or more target brain structures, in accordance with some embodiments of the present invention. Reference is also made to FIG. 2, which is a flowchart of a method of annotation and/or reconstruction of one or more target brain structures, in accordance with some embodiments of the present invention. Reference is also made to FIG. 3, which is a schematic depicting a slice of a 3D Tl weighted MRI scan with automatically identified landmarks, in accordance with some embodiments of the present invention. Reference is also made to FIG. 4, which is a schematic depicting a reorientation of the 3D Tl weighted MRI scan, in accordance with some embodiments of the present invention. Reference is also made to FIG. 5, which is a schematic of a whole brain reconstruction 502 that includes segmentation and parcellation of brain structures, in accordance with some embodiments of the present invention. Reference is also made to FIG. 6, which is a schematic of a thalamic parcellation 602, in accordance with some embodiments of the present invention. Reference is also made to FIG. 7, which is a schematic of an image 702 depicting both a cerebellum 704 with dentate nucleus 706 and parcellated thalamus with nuclei 708 including a VIM 710, in accordance with some embodiments of the present invention. Reference is also made to FIG. 8, which is a schematic 802 depicting isolated white matter fibers of the DRTT 804 connecting between a dentate nucleus 806 of the cerebellum to the contralateral VIM nucleus 808 of the thalamus, in accordance with some embodiments of the present invention. Reference is also made to FIG. 9, which is a schematic of an exemplary 3D reconstruction 902, in accordance with some embodiments of the present invention. Reference is also made to FIG. 10, which is a schematic 1002 of subject specific masks 1010 of the VIM and VC, and of the DRTT registered and merged together with a T1 weighted scan, in accordance with some embodiments of the present invention. Reference is also made to FIG. 11, which includes graphs presenting results of the experiment comparing classic stereotactic targeting and personalized integrative targeting, in accordance with some embodiments of the present invention. Reference is also made to FIG. 12, which includes graphs presenting results of adverse effects in an experiment comparing classic stereotactic targeting and personalized integrative targeting, in accordance with some embodiments of the present invention. Reference is also made to FIG. 13, which is a schematic depicting preliminary data of pre-and post-surgical scans which undergone the subject-specific 3D reconstruction approaches described herein, in accordance with some embodiments of the present invention.

System 100 may execute the acts of the method described with reference to FIG. 2-12, for example, by a hardware processor(s) 102 of a computing device 104 executing code 106A stored in a memory 106.

Computing device 104 receives medical images, which may be captured by medical imaging devices(s) 112. The images captured by medical imaging devices(s) 112 may be stored in an image repository 114, for example, data storage device 122 of computing device 104, a storage server 118, a data storage device, a computing cloud, virtual memory, and a hard disk. Computing device 104 generates a 3D reconstruction of a target brain structure using the medical images, as described herein.

Computing device 104 may be implemented as, for example, a radiology workstation, a surgical workstation, a client terminal, a virtual machine, a server, a virtual server, a computing cloud, a group of connected devices, a mobile device, a desktop computer, a thin client, a kiosk, and a mobile device (e.g., a Smartphone, a Tablet computer, a laptop computer, a wearable computer, glasses computer, and a watch computer). Computing device 104 may include an advanced add-on to a radiology workstation and/or a surgical workstation for presenting the reconstructed target brain structure(s), for example, for performing automated targeted brain surgery and/or assisting in a targeted brain surgical procedure and/or for guiding a focused ultrasound ablation system and/or a stereotactic radiosurgery system to specific target structures of the brain.

Multiple architectures of system 100 based on computing device 104 may be implemented. For example:
Computing device 104 executing stored code instructions 106A, may be implemented as one or more servers (e.g., network server, web server, a computing cloud, a virtual server) that provides services (e.g., one or more of the acts described with reference to FIG. 1) to one or more client terminals 108 (e.g., remotely located remote surgical workstation) over a network 110. For example, providing software as a service (SaaS) to the client terminal(s) 108, providing software services accessible using a software interface (e.g., application programming interface (API), software development kit (SDK)), providing an application for local download to the client terminal(s) 108, providing an add-on to a web browser running on client terminal(s) 108, and/or providing functions using a remote access session to the client terminals 108, such as through a web browser executed by client terminal 108 accessing a web sited hosted by computing device 108. For example, client terminals access code 106A running on computing device 104 via web browsers running on the client terminals, client terminals download code 106A for local execution (e.g., for execution within a surgical planning application running on a workstation), a plug-in that runs and/or accesses code 106A is installed on the web browser running on the client terminals, and/or client terminals use an API to access code 106A running on computing device 104. In such implementation, users using client terminals 108 provide the medical images, and obtain a reconstruction of the target brain structure, as described herein.
Computing device 104 may be implemented as a standalone device (e.g., surgical planning workstation, kiosk, client terminal, smartphone, server) that includes locally stored code instructions 106A that implement one or more of the acts described with reference to FIGS. 2-12. The locally stored instructions may be obtained from another server (e.g., 118), for example, by downloading the code over the network, and/or loading the code from a portable storage device. In such implementation, each user uses their own computing device 104 to locally select the images for generating the reconstruction of the target brain structure(s), as described herein.
Computing device 104 may be integrated within treatment device 101, for example, as code installed on a workstation associate with treatment device 101, and/or in network communication with treatment device 101. For example, surgeons using treatment device 101 to plan stereotactic ultrasound and/or radiosurgery on a brain of a subject use code 106A to obtain the reconstruction of the target brain structure which is to be targeted by the treatment device 101.

Medical imaging devices 112 may be referred to as anatomical imaging devices and/or imaging modalities. Medical imaging devices 112 capture medical and/or anatomical images of subjects, depicting internal tissues of the brain. Medical imaging devices 112 may capture 3D images, 3D datasets, and/or 2D images and/or 2D datasets where the 2D images may be associated with 3D data and/or used to generate 3D images. Exemplary medical imaging device(s) 112 include: a magnetic resonance imaging (MRI) device, an ultrasound machine (e.g., 3D), a CT machine, and/or a nuclear imaging machine. Medical imaging devices 112 may be operated under different imaging protocols, to obtain the different images described herein. For example, the same MRI machine may generate T1 weighted images, T2 weighted images, diffusion tractography images, and proton density (PD) images.

Hardware processor(s) 102 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include one or more processors (homogenous or heterogeneous), which may be arranged for parallel processing, as clusters and/or as one or more multi core processing units.

Memory 106 (also referred to herein as a program store, and/or data storage device) stores code instruction for execution by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 106 stores code 106A that implements one or more acts and/or features of the method described with reference to FIGS. 2-12.

Computing device 104 may include a data storage device 122 for storing data, for example, the obtained images, and/or treatment code 112A which generates instructions for a treatment device 101 for automatic treatment of the target brain structure using the reconstructions (e.g., focused ultrasound), and/or image analysis code 122B which performs image processing on the images for generating outcomes used for the reconstructions, for example, tractography, segmentation, and the like as described herein. Data storage device 122 may be implemented as, for example, a memory, a local hard-drive, a removable storage device, an optical disk, a storage device, and/or as a remote server and/or computing cloud (e.g., accessed over network 110). It is noted that code 122A-B may be stored in data storage device 122, with executing portions loaded into memory 106 for execution by processor(s) 102.

Computing device 104 may receive images 116 (e.g., captured by medical imaging device(s) 112) using one or more imaging interfaces 120, for example, a wire connection (e.g., physical port), a wireless connection (e.g., antenna), a local bus, a port for connection of a data storage device, a network interface card, other physical interface implementations, and/or virtual interfaces (e.g., software interface, virtual private network (VPN) connection, application programming interface (API), software development kit (SDK)).

Computing device 104 may include data interface 124, optionally a network interface, for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations.

It is noted that imaging interface 120 and data interface 124 may exist as two independent interfaces (e.g., two network ports), as two virtual interfaces on a common physical interface (e.g., virtual networks on a common network port), and/or integrated into a single interface (e.g., network interface).

Computing device 104 may communicate using network 110 (or another communication channel, such as through a direct link (e.g., cable, wireless) and/or indirect link (e.g., via an intermediary computing device such as a server, and/or via a storage device) with one or more of:
Server(s) 118, for example, to obtain images 116, and/or obtain an updated version of code 106A and/or code 122A-B.
Client terminal(s) 108, for example, when computing device 104 acts as a server providing services to the client terminals 108 for reconstruction of target brain structures for different subjects.
Image repository 114 that stores images 116 captured by imaging sensor(s) 112.
Treatment device 101 that administers treatment to the reconstructed target brain structures, for example, focused ultrasound, and/or stereotactic radiosurgery.

Computing device 104 and/or client terminal(s) 108 includes or is in communication with a physical user interface 126 that includes a mechanism designed for a user to enter data and/or view data. Exemplary physical user interfaces 126 include, for example, one or more of, a touchscreen, a display, a keyboard, a mouse, and voice activated software using speakers and microphone.

Referring now back to FIG. 2, the features of the method are described broadly, for different applications and/or different treatments. An exemplary use case for targeted treatment of the ventral inter-mediate (VIM) nucleus of the thalamus is described as a not necessarily limiting application.

At 202, multiple images depicting at least a portion of a brain of a subject are obtained (e.g., received, accessed, provided). The images may be scans of a head of the subject. The images may be anatomical and/or functional images. The images may be 3D images, for example, presented as 2D image slices and/or other planes of 3D images.

The images may include a first image captured using a first protocol, a second image captured using a second protocol different than the first protocol, and a third image captured using a third protocol different than the first protocol and the second protocol. The first protocol may be designed for contrast between gray matter and white matter of the brain. The second protocol may be designed for segmentation of the at least one brain structure. The third protocol may be designed for tractography. The image may be 3D MRI images. For example, the first image may be a T1 weighted MRI image, the second image may be a T2 weighted MRI image, and/or the third image may be a diffusion tensor image (DTI) and/or diffusion-weighted image.

In an exemplary use case, the T1 weighted scan may be used as the background layer and/or reference image of the reconstructed model, where analyses results may be presented for higher visual resolution. The T2 weighted scan may be used for intra-thalamic segmentation. The DTI scan may be used for reconstruction of white matter fibers of the DRTT connecting the dentate nucleus of the cerebellum through the red nucleus of the midbrain and finally to the VIM of the thalamus.

It is noted that other imaging modalities and/or protocols may be used, for example, FGATIR, WAIR, PD, and the like.

Features 204-208 represent optional exemplary pre-processing features.

At 204, the processor may execute code for automatically detecting one or more landmarks, optionally stereotactic landmarks, in proximity to and/or external to the target brain structure. The landmarks may be automatically detected in the realigned first image.

The automatic detection may be done, for example, by feeding the image into a detector neural network trained on a training dataset of images labelled with ground truth indications, and/or other image processing approaches such as feature extraction, based on detection of shapes of pixel intensity regions, and the like.

In the exemplary use case, the stereotactic landmarks may include the anterior commissure (AC), posterior commissure (PC), and/or third ventricle.

The alignment may be done, for example, using a linear realignment process. In the exemplary use case, the alignment may be done using an Inter-Commissural Line (ICL) as an anterior-posterior axis.

The detected landmarks may be set as an origin of the first image. In the exemplary use case, the AC or PC may be defined as the first image axes origin.

A volume of the brain may be aligned so that the stereotactic landmarks are set on a same axial plane as the origin. In the exemplary use case, the brain volume is aligned so that PC landmark is set on the same axial plane as the AC.

The described approach may automatically identify the landmarks, optionally the AC and/or PC, in an objective and repeatable manner.

Referring now back to FIG. 3, scan 302 depicting AC 304, PC 306. AC 304 and PC 306 are landmarks that are automatically detected. Origin 304 or 306 may be automatically set.

Referring now back to FIG. 4, images 402 are from scan 302 of FIG. 3. Images at a top row 404 depicts the initial scan. Crosshairs 406 represent the origin of the initial scan. Images at a bottom row 408 depict the state of the scan after AC-PC reorientation. Crosshairs 410 represent AC image origin.

Referring now back to FIG. 2, at 206, the processor may execute code for aligning the first image (e.g., 3D T1 weighted scan) to a right-anterior-superior (RAS) space, for example, using a linear realignment process.

Referring now back to FIG. 2, at 208, the processor may execute code for co-registering the second image (e.g., T2 weighted scan) to the aligned first image (e.g., aligned T1 weighted scan) serving as reference. The registration may be done using the landmarks. The registration generates the same dimensions and/or orientation for the two images.

At 210, the processor may execute code for reconstructing at least a part of the brain, optionally the whole brain. The aligned and centered first anatomical image (e.g., AC-PC aligned and centered 3D T1 weighted image) may be fed as input into a reconstruction process that performs the reconstruction.

The reconstruction includes boundaries of brain structures that include the target brain structure. The reconstruction is insufficient for parcellation of the target brain structure, since the target brain structure is of the same type of tissue as other neighboring brain structures. The target brain structure cannot be visually differentiated from the other neighboring brain structures since the target brain structure looks similar to the other neighboring brain structures. For example, the VIM nucleus of the thalamus cannot be visually delineated from other nuclei of the thalamus since they are all gray matter, which appear very similar on the image(s).

An exemplary not necessarily limiting reconstruction approach is now described. The aligned and centered first anatomical image may be used as an input for the reconstruction process, for example, Freesurfer's (v7.0.0) cortical and sub-cortical reconstruction process, for example, as described with reference to Fischl B. FreeSurfer. Neuroimage 2012; 62: 774-81. doi: 10.1016/j.neuroimage.2012.01.021 incorporated herein by reference in its entirety. A complete labeling of cortical sulci and gyri surface may be performed by assigning a neuroanatomical label to each location on a cortical surface model. The main steps of processing structural MRI data include Skull stripping, gray-white matter segmentation, reconstruction of gray-white boundary surface and pial surface, labelling of regions on the cortical surface and subcortical brain structures, and parcellating the cortical surface into gyral based ROIs. This analysis shows the boundaries and volumes of all labeled brain structures such as the cerebellum and thalamus, however, it is not sufficient for discriminating the intra thalamic/cerebellar nuclei.

Referring now back to FIG. 5, whole brain reconstruction 502, that includes segmentation and parcellation of brain structures, is presented. It is noted that thalamic nuclei or other sub-structures cannot be visually delineated.

Referring now back to FIG. 2, at 212, the processor may execute code for reconstructing and/or parceling the target brain structure(s). The target brain structure is optionally parcellated in the second image (e.g., the co-registered T2 weighted scan), optionally using a reference atlas.

Optionally, a full reconstruction of the entire brain with segmentation and parcellation of gray and white matter on a surface mesh is computed, designed for maximal accuracy and/or sub-millimeter precision.

The target brain structure may be segmented according to the second image registered to the first image (e.g., the co-registered T2 weighted scan).

Non-target brain structure(s) located in proximal to and external to the target brain structure, may be segmented. The target brain structure(s) and the non-target brain structure(s) may be of a same matter type and/or may be sub-structures within a main structure.

In the exemplary use case, the target brain structure is the VIM, and the non-target brain structure(s) may be a ventral caudal (VC) nucleus and/or nearby brain tissue within the thalamus and/or nearby tissue such as the internal capsule located externally to the thalamus, that is at risk for unintended damage during treatment of the VIM.

The non-target brain structure(s) and the target brain structure(s) may be cropped, creating cropped images. The cropped images may be resliced to dimensions of the aligned first image. The resliced cropped images may be overlayed upon the first image. Boundaries of the non-target brain structure and the target brain structure are depicted in the overlay.

In the exemplary use case, the co-registered T2 weighted scan is used for parcellation of the inner thalamic nuclei, following the probabilistic ex-vivo atlas for thalamic parcellation, for example, by Iglesias, for example, as described with reference to Iglesias J E, Insausti R, Lerma-Usabiaga G, et al. A probabilistic atlas of the human thalamic nuclei combining ex vivo MRI and histology. Neuroimage 2018; 183: 314-26. doi: 10.1016/j.neuroimage.2018.08.012 incorporated herein by reference in its entirety. From the reconstructed thalamic nuclei image, the Ventral Lateral Posterior nucleus (Vlp, otherwise known as the VIM) and VC located posterior to it, are cropped. The cropped image of these nuclei maybe resliced to the dimensions of the aligned T1 weighted scan and may be overlaid upon the T1 weighted scan so the VIM, VC, and their contour lines are visible in 3D.

Referring now back to FIG. 6, an axial view 604 and a sagittal view 606 of reconstructed and/or parcellated thalamus 608 are shown. The images slices shown as axial view 604 and sagittal view 606 are from a 3D T1 weighted MRI scan. It is noted that the overlaid thalamic parcellation 608 is from the T2 weighted MRI scan.

Referring now back to FIG. 2, at 214, the processor may execute code for segmenting and/or parceling originating brain structure(s) from the anatomical image and/or the reconstruction image and/or using a reference atlas. For example, in the exemplary use case, the originating brain structure is one or both Dentate nuclei.

A main brain structure that includes the originating brain structure therein may be isolated from the reconstruction and/or from the anatomical scan. For example, in the exemplary use case, the main brain structure is the cerebellum. A transformation matrix may be computed for registering the isolated main brain structure to a space of an atlas. The atlas may be inversely transformed using the transformation matrix into a structural reconstruction space that includes the segmented and parcellated originating brain structure. The 3D reconstruction space may correspond to, and/or be mapped to, and/or be transformed to the anatomical native space.

It is noted that the term originating brain structure may be substituted with the term terminating brain structure, and/or connected brain structure. The target brain structure and/or originating brain structure (and/or terminating brain structure, and/or connected brain structure) may be gray matter, connected to each other via white matter tracts. It is noted that the flow of information along the white matter tract may be uni-directional, and/or bi-directional. The terms originating brain structure, term terminating brain structure, and/or connected brain structure, are not meant to limit the type of connecting white matter tract based on the direction of flow of information.

An exemplary approach, such as for the exemplary use case, is now described. The dentate nucleus of the cerebellum ipsilateral to the tremorous limb may be parcellated, for example, using SUIT (Spatially Unbiased Infra-Tentorial) atlas, for example, as described with reference to Diedrichsen J. Representational Models. 2006, incorporated herein by reference in its entirety, and/or toolbox. The cerebellum of the T1 weighted scan, which is outputted from the cortical and subcortical reconstruction process (e.g., as described with reference to 210), is isolated and/or segmented. The isolated cerebellum is registered to the SUIT atlas space and the transformation matrix is computed. The SUIT atlas is inversely transformed using the computed matrix back into the structural reconstruction space, resulting in a parcellated cerebellum. The parcellated and segmented output of the structural reconstruction is merged with the parcellated thalamic nuclei (e.g., as described with reference to 212) and dentate nuclei from the parcellated cerebellum (as described herein). The combined parcellation image is used in the tractography creation of the DRTT. i.e., the reconstruction of the white matter tracks stemming from dentate nucleus to the VIM nucleus, as described herein, which may enable generating the 3D reconstruction that includes the white matter tract(s) and target brain structure(s) and optionally the originating brain structure(s) within the anatomical native space.

Referring now back to FIG. 7, image 702 depicts both cerebellum 704 with dentate 706 nuclei and thalamus 708 with nuclei including VIM 710.

Referring now back to FIG. 2, at 215, the processor may execute code for creating a tractogram of at least the part of the brain, optionally the whole brain. Optionally, the tractogram that is created of the whole brain includes a connectome, for example, computed as described with reference to U.S. Application No. 63/213,242, titled “METHOD AND SYSTEM FOR DETERMINING CONDITION OF A SUBJECT BASED ON CONNECTOME”, filed on Jun. 22, 2021, by at least one common author of the instant application, incorporated herein by reference in its entirety.

Referring now back to FIG. 2, at 216, the processor may execute code for filtering white matter fibers of the tractography, optionally of the connectome, for isolating one or more white matter tracts that connect the originating brain structure(s) and the target brain structure(s). The white matter fibers may be identified and filtered on the third image. In the exemplary use case, the white matter tract is the DRTT, which extends from the dentate nucleus to the VIM of the thalamus. The DRTT may be reconstructed in the diffusion native space stemming from the dentate nucleus, which was automatically segmented out of the Tl weighted scan, to the thalamic VIM.

The termination location of the white matter fibers that also connect to the originating brain structure(s) provides an accurate location of the target brain structure. During the reconstruction, the identified target white matter fibers may correct the location of the target brain structure(s) from the location defined by the reference atlas to the personalized location in the anatomy of the subject, based on the fact that the target white matter fibers physically connect to the target brain structure(s).

An exemplary approach, such as for the exemplary use case, is now described. The third image, which may include DTI data, may preprocessed, for example, by a standard DWI preprocessing pipeline, such as utilizing MRtrix3, for example, as described with reference to Tournier J-D, Smith R, Raffelt D, et al. MRtrix3: A fast, flexible and open software framework for medical image processing and visualisation. Neuroimage 2019; 202: 116137. doi: 10.1016/j.neuroimage.2019.116137 incorporated herein by reference in its entirety, and FSL, for example, as described with reference to Smith S M, Jenkinson M, Woolrich M W, et al. Advances in Functional and Structural MR Image Analysis and Implementation as FSL Technical Report TR04SS2.;: 1-15 incorporated herein by reference in its entirety. Data may be denoised and/or corrected for eddy currents and/or motion artefacts using FSL's top-up, and un-warped using for single shell with no Phase-AP correction. The first image (e.g., T1 weighted image) is co-registered into the DWI space. The brain area is extracted from the DWI image, for example, using “bet” by Advanced normalization tools, for example, as described with reference to Avants B B, Tustison N, Hans J. Advanced Normalization Tools (ANTS). 2014;: Section 16.2 incorporated herein by reference in its entirety. Basis function may be derived, for example, using Tournier's method for Single shell acquisition. Constrained spherical deconvolution may be performed to calculate fiber orientation density (FOD) for each voxel. The first image (e.g., structural T1 weighted image) may be segmented into tissue type segmentation, for example, into the following 5 types; cortical and sub cortical gray matter, white matter, CSF, and pathological tissue.

Tractography may be calculated, for example, using MRtrix3 anatomically constrained tractography (ACT) to generate probabilistic streamlines out of the calculated FODs. Using this approach, two versions of streamlines may be created, for example: about 10 million streamlines. Streamlines are refined using spherical deconvolution informed filtering of tractograms (SIFT2) model. A whole brain connectome may be constructed. Streamlines may be filtered to create an image that includes only those that cross through the masks of the ipsilateral dentate nucleus and the contralateral VIM (created for example as described with reference to 212-214) reconstructing the DRTT.

Referring now back to FIG. 8, isolated white matter fibers of the DRTT 804 connecting between dentate nucleus 806 of the cerebellum to VIM nucleus 808 of the thalamus, which are computed as described herein, are depicted.

Referring now back to FIG. 2, at 218, the processor may execute code for creating a 3D reconstruction that includes at least the target brain structure(s) and the target white matter tract(s), optionally within an anatomical native space, for example, within a set of virtual 3D coordinates that correspond to real-life physical space The locations of the target brain structure(s) and the target white matter tract(s) may be determined simultaneously and accurately within the anatomical native space.

Optionally, the target white matter fibers(s) (e.g., DRTT) which may be reconstructed in the diffusion native space (e.g., as described with reference to 216), may be mapped and/or transformed to the anatomical native space.

The 3D reconstruction may further include the originating brain structure, optionally within the anatomical native space.

The target brain structure(s) and/or the target white matter tract(s) and/or the originating brain structure(s) may be transformed and/or mapped to the anatomical native space, and/or transformed and/or mapped to different spaces to enable creating the 3D reconstruction that includes the target brain structure(s) and/or the target white matter tract(s) and/or the originating brain structure(s). Exemplary transformations and/or mappings are, for example, as described herein.

The 3D reconstruction may be created by overlaying the structures and/or tracts on a background structural scan which may be defined by the anatomical native space. A location indicating the automatically detected stereotactic landmark may be marked on the 3D reconstruction.

An exemplary approach, such as for the exemplary use case, is now described. The 3D model may be created as a visualization of the thalamic nuclei, contralateral dentate nucleus of the cerebellum and DRTT, which are overlaid upon a Tl weighted, PD or T2 weighted image. The reconstructed model may present the indirect surgical target derived from automatically detected stereotactic landmarks (e.g., AC, PC, 3rd ventricle) as described herein.

Referring now back to FIG. 9, schematic 902 is a 3D reconstruction (also referred to as visualization model) depicting thalamic nuclei 904, contralateral dentate nucleus of the cerebellum (not shown in the image), and DRTT 906 overlaid upon a T1 weighted MRI image. A sagittal view 908, a coronal view 910, and an axial view 912 are shown. Crosses 914 mark the optimal surgical site for treatment of the VIM nucleus of the thalamus, for the exemplary use case.

Referring now back to FIG. 2, alternatively or additionally to 218, at 220 the processor may execute code for creating a single combined 3D image by combining the 3D reconstruction of the target brain structure and/or the target white matter tract and/or the originating brain structure, with a background depicting a structure of the brain. The single combined 3D image may be designed to be compatible with medical standards and software for storing and/or presenting and/or processing of medical images, for example DICOM, PACS, and the like. The single combined 3D image may be designed to be compatible with standards being developed for treatment planning and/or guidance, such as using MRgHIFU. The reconstruction of the part of the brain with the segmented target brain structure may be merged with the segmented originating brain structure and with the filtered white-matter tracts to create a merged image.

The 3D image(s) and/or 3D reconstruction(s) may be provided, for example, presented on a display, stored in a data storage device (e.g., on a removal storage, by a PACS server and/or other digital image archiving system), fed into another process (e.g., to a controller for delivery of treatment according to the 3D image and/or 3D reconstruction), forward to another computing device (e.g., over a network), and/or otherwise provided for pre-surgical and/or real-time fusion.

An exemplary approach, such as for the exemplary use case, is now described. The 3D image(s) and/or 3D reconstruction(s) may include subject-specific masks of the VIM and optionally VC, and of the DRTT registered and merged together with a common structural scan used for planning (e.g., T1 weighted, T2 weighted, PD, and the like). The 3D image(s) and/or 3D reconstruction(s) is designed to be compatible with the treatment center's (e.g., hospital's) digital images archiving system (e.g., PACS), allowing pre-surgical and/or real-time fusion with other scans (e.g., during surgery) and/or integration with other medical systems.

At 222, the 3D reconstruction may be provided, for example, presented on a display, stored on a data storage device, forwarded to another computing device, and/or fed into another process such as an automated image guidance process.

Optionally, the 3D reconstruction is fed into a guidance system for neural intervention for image guided treatment of the target brain structure(s) guided by the 3D reconstruction. Optionally, the subject depicted in the generated 3D image(s) and/or 3D reconstruction(s) may be treated based on the generated 3D image(s) and/or 3D reconstruction(s), optionally by the image guided system for neural intervention. An invasive and/or non-invasive treatment may be applied to the target brain structures depicted in the 3D image(s) and/or 3D reconstructions, with high accuracy, optionally automatically and/or semi-automatically by the guidance system. In the use case, the subject may be treated for essential tremor by applying focused ultrasound to the VIM under image guidance based on the 3D image(s) and/or 3D reconstruction(s).

Alternatively or additionally, the 3D reconstruction may be projected via an artificial reality and/or virtual reality platform, for example, presented within a virtual headset.

Referring now back to FIG. 10, subject specific masks 1010 of the VIM and VC, and of the DRTT registered and merged together with a T1 weighted scan, are shown. Sagittal 1004, coronal 1006, and axial view 1008 are shown.

Various embodiments and/or aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in at necessarily non limiting fashion.

Inventors conduced a preliminary study to examine the efficacy of a personalized integrative targeting system based on embodiments described herein.

Participants: Fifty-one essential tremor patients with a mean age of 71 (8.32) were included in the analyses, of which 34 undergone MRgHIFU surgery based on the classic stereotactic targeting and 17 patients had undergone MRgHIFU surgery based on the personalized integrative targeting system based on embodiments described herein.

Methods: Number of sonications and number of ablations were documented during surgery. Independent sample t-tests and chi square goodness of fit test were conducted to examine the difference in number of sonications and ablations as well as to determine whether the proportion of adverse effects differed when using the two different targeting systems. Significance threshold was set as p<0.05.

Results: Patients who were operated using the classic stereotactic targeting had significantly larger number of sonications (mean(sd)=9.971(4.174)), and a larger number of ablations (mean(sd)=3.794(2.026)) compared to the number of sonications (mean(sd)=6.000(2.715)) and ablations (mead(sd)=2.353(1.366)) done using the personalized integrative targeting system based on embodiments described herein (p=0.000,p=0.004, respectively). The proportion of sensory adverse effects and gait disturbance differed by targeting system (χ²(1, N=27) =5.082, p=0.034; χ² (1, N=27)=4.201, p=0.049; respectively). Patients were less likely to develop adverse effects using the personalized integrative targeting system than from using the classic stereotactic targeting.

Referring now back to FIG. 11, the graphs present results of the experiment comparing classic stereotactic targeting and personalized integrative targeting based on embodiments described herein. Graph 1102 shows a significant decrease in number of sonications using personalized integrative targeting (based on embodiments described herein) in comparison to classic stereotactic targeting. Graph 1104 shows a significant decrease in number of ablations using personalized integrative targeting (based on embodiments described herein) in comparison to classic stereotactic targeting.

Referring now back to FIG. 12, graphs 1202 and 1204 present results of adverse effects of an experiment comparing classic stereotactic targeting based on standard approaches and personalized integrative targeting based on embodiments described herein. Graph 1202 presents results of sensory adverse effects. As shown in graph 1202, for classic stereotactic approaches, 6 cases reported sensory adverse effects, and 4 cases did not report sensory adverse effects. In contrast, for a personalized integrative approach based on embodiments described herein, 3 cases reported sensory adverse effects, and 14 cases did not report sensory adverse effects. Graph 1204 presents results of gait disturbance adverse effects. As shown in graph 1204, for classic stereotactic approaches, 7 cases reported gait disturbance adverse effects, and 3 cases did not report gait disturbance adverse effects. In contrast, for a personalized integrative approach based on embodiments described herein, 5 cases reported gait disturbance adverse effects, and 12 cases did not report gait disturbance adverse effects. The results presented in graphs 1202 and 1204 provide evidence that the personalized integrative approach based on embodiments described herein is less likely to result in adverse events in comparison to classic stereotactic approaches.

Reference now back to FIG. 13, schematic 1302 depicts the 3D reconstruction for treatment generated using embodiments described herein, prior to treatment, used for surgical planning. Schematic 1304 depicting a post-surgical 3D reconstruction computed using post-surgical scans using embodiments described herein. A lesion 1306 located in the intersection between DRTT 1308 and the VIM nuclei 1310 has been generated using MRgHIFU using the 3D reconstructions generated using approaches described herein. Lesion 1306 in post-surgical reconstruction 1304 is shown as lack of connectivity between DRTT 1308 and VIM nuclei 1310, in comparison to pre-surgical reconstruction 1302 where the DRTT is shown connected to the VIM nuclei.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant images will be developed and the scope of the term image is intended to include all such new technologies a priori.

As used herein the term “about” refers to '10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A computer implemented method of reconstruction of at least one target brain structure, comprising, using at least one processor for processing a plurality of images depicting a brain of a subject by:
reconstructing at least a part of the brain comprising boundaries of a plurality of brain structures that include the at least one target brain structure, wherein the reconstruction is insufficient for parceling of the at least one target brain structure into sub-structures of a same type of gray or white matter;
reconstructing and parceling the at least one target brain structure using a reference atlas;
segmenting and parceling at least one originating brain structure using the reference atlas;
filtering white matter fibers to isolate at least one target white matter tract connecting the at least one originating brain structure and the at least one target brain structure; and
creating a 3D reconstruction of the at least one target brain structure and the at least one target white matter tract, wherein the at least one target white matter tract and the at least one target brain structure are transformed and/or mapped to an anatomical native space.

2. The computer implemented method of claim 1, further comprising:
creating a single combined 3D image by combining the 3D reconstruction of the at least one target brain structure and the at least one target white matter tract with the at least one originating brain structure transformed and/or mapped into the anatomical native space and with a background depicting a structure of the brain defined within the anatomical native space, wherein the single combined 3D image is compatible with medical archive standards for storing and presenting 3D medical images.

3. The computer implemented method of claim 1, further comprising:
automatically detecting at least one stereotactic landmark in proximity to and external to the at least one target brain structure;
realigning a first image to (right-anterior-superior) RAS space using linear realignment, wherein the at least one stereotactic landmark is automatically detected in the realigned first image and set as an origin of the first image; and
aligning a volume of the brain so that the at least one stereotactic landmarks are set on a same axial plane as the origin.

4. The computer implemented method of claim 3, wherein the at least one stereotactic landmark is selected from a group comprising: anterior commissure (AC), posterior commissure (PC), and third ventricle, and wherein aligning comprises aligning an Inter-Commissural Line (ICL) as an anterior-posterior axis.

5. The computer implemented method of claim 3, further comprising:
wherein the at least one stereotactic landmark is of a first anatomical image in proximity to and external to the at least one target brain structure; and
co-registering at least one second image to an aligned first anatomical image using the at least one stereotactic landmark to obtain same image dimensions and/or orientation.

6. The computer implemented method of claim 5, further comprising feeding an aligned and centered anatomical image as input into a reconstruction process that reconstructs the part of the brain, wherein the part of the brain comprises a whole brain.

7. The computer implemented method of claim 3, further comprising marking a location indicating the at least one stereotactic landmark on the 3D reconstruction.

8. The computer implemented method of claim 1, wherein the at least one target brain structure comprises at least one ventral intermediate nucleus (VIM) of a thalamus, and the at least one originating brain structure comprises at least one Dentate nucleus.

9. The computer implemented method of claim 1, further comprising segmenting the at least one target brain structure according to a second image registered to the first image, wherein the second image is captured using a second protocol different than a first protocol used to capture the first image.

10. The computer implemented method of claim 9, wherein segmenting comprises segmenting at least one non-target brain structure located in proximal to and external to the at least one target brain structure.

11-12. (canceled)

13. The computer implemented method of claim 10, further comprising cropping the at least one non-target brain structure and the at least one target brain structure to create cropped images, reslicing the cropped images to dimensions of an aligned first image, and overlaying the resliced cropped images upon the first image, wherein boundaries of the at least one non-target brain structure and the at least one target brain structure are depicted in the overlay.

14. The computer implemented method of claim 1, wherein segmenting at least one originating brain structure comprises isolating and segmenting from the anatomical scan a main brain structure that includes the originating brain structure therein, computing a transformation matrix for registering the isolated main brain structure to a space of an atlas, inversely transforming the atlas using the transformation matrix into a structural reconstruction space comprising the segmented and parcellated at least one originating brain structure.

15. The computer implemented method of claim 1, further comprising merging the reconstruction of the part of the brain with the segmented at least one target brain structure and the segmented at least one originating brain structure to create a merged image.

16. The computer implemented method of claim 1, wherein creating the 3D reconstruction comprises overlaying the at least one target brain structure and overlaying at least one of: the at least one originating brain structure, and the at least one target white matter tract, on a background anatomical scan and within the anatomical native space.

17. The computer implemented method of claim 1, wherein the at least one target brain structure comprises a VIM of a thalamus, and further comprising treating the subject for essential tremor by applying focused ultrasound to the VIM.

18. The computer implemented method of claim 1, further comprising feeding the 3D reconstruction into a guidance system for neural intervention for image guided treatment of the at least one target brain structure guided by the 3D reconstruction.

19. The computer implemented method of claim 1, wherein the plurality of images comprise a first image captured using a first protocol, a second image captured using a second protocol different than the first protocol, and a third image captured using a third protocol different than the first protocol and the second protocol.

20-22. (canceled)

23. The computer implemented method of claim 1, wherein the at least one target brain structure comprises at least one VIM of a thalamus, the at least one originating brain structure comprises at least one Dentate nucleus, and the at least one white matter tract comprises a Dento-Rubro Thalamic Tract (DRTT) extending from the at least one Dentate nucleus to the VIM.

24. A system for reconstruction of at least one target brain structure, comprising:
at least one processor executing a code for processing a plurality of images depicting a brain of a subject by:
reconstructing at least a part of the brain comprising boundaries of a plurality of brain structures that include the at least one target brain structure, wherein the reconstruction is insufficient for parceling of the at least one target brain structure into sub-structures of a same type of gray or white matter;
reconstructing and parceling the at least one target brain structure using a reference atlas;
segmenting and parceling at least one originating brain structure using the reference atlas;
filtering white matter fibers to isolate at least one target white matter tract connecting the at least one originating brain structure and the at least one target brain structure; and
creating a 3D reconstruction of the at least one target brain structure and the at least one target white matter tract, wherein the at least one target white matter tract and the at least one target brain structure are defined within an anatomical native space.

25. A non-transitory medium storing program instructions for reconstruction of at least one target brain structure, which, when executed by at least one processor, cause the at least one processor to:
process a plurality of images depicting a brain of a subject by:
reconstructing at least a part of the brain comprising boundaries of a plurality of brain structures that include the at least one target brain structure, wherein the reconstruction is insufficient for parceling of the at least one target brain structure into sub-structures of a same type of gray or white matter;
reconstructing and parceling the at least one target brain structure using a reference atlas;
segmenting and parceling at least one originating brain structure using the reference atlas;
filtering white matter fibers to isolate at least one target white matter tract connecting the at least one originating brain structure and the at least one target brain structure; and
creating a 3D reconstruction of the at least one target brain structure and the at least one target white matter tract, wherein the at least one target white matter tract and the at least one target brain structure are defined within an anatomical native space.
